(19) United States
(12) Patent Application Publication
(10) Pub. No.: US 2021/0299170 A1
(43) Pub. Date: Sep. 30, 2021

(54) METHOD FOR IMPROVING ANTICANCER AGENT EFFICACY

(71) Applicant: DA VOLTERRA, Paris (FR)

(72) Inventor: Jean DE GUNZBURG, London (GB)

(21) Appl. No.: 17/263,783

(22) PCT Filed: Aug. 5, 2019
(86) PCT No.: PCT/EP2019/071024

§ 371 Date: Jan. 27, 2021

(30) Foreign Application Priority Data
Aug. 5, 2018 (EP) 18187408.2

(51) Int. Cl. A61K 33/44; A61K 45/06; A61K 38/50; A61K 38/46; A61P 35/00; A61K 9/50

(52) U.S. Cl. CPC A61K 33/44; A61K 45/06; A61K 38/50; A61K 38/465; A61K 9/0053; A61K 9/5036; C12Y 305/02006; C12Y 301/01; A61P 35/00

(57) ABSTRACT

The present invention relates to a method for improving the therapeutic efficacy of an anticancer agent, comprising administering to a subject in need thereof an effective amount of an adsorbent or an antibiotic-inactivating enzyme.

### FIELD OF THE INVENTION

The present invention pertains to the field of therapy. More particularly, it is herein disclosed a method for improving the therapeutic efficacy of an anticancer agent, comprising administering to a subject in need thereof an effective amount of an adsorbent. In particular, the present invention can be used to prevent the disruption of the intestinal microbiota in a subject and improve thereby the therapeutic efficacy of an anticancer agent administered to the subject in need thereof.

### BACKGROUND OF THE INVENTION

Cancer is characterized by the uncontrolled growth of cells in the body, leading to the invasion of essential organs and often death. Initially, the pharmacological treatment of cancer utilized non-specific cytotoxic agents that targeted all rapidly dividing cells, including normal cells. These non-specific cytotoxic agents have anti-tumor effects but their use is often limited by severe toxicities and they often fail to cure the patients in a durable manner. As the understanding of the proteins and pathways that enable cancer cells to thrive has evolved, newer more targeted agents have been developed that block specific proteins that are activated in cancer cells.

In particular, immuno-oncology agents were developed: they use the patient's immune system to help treat cancer. The immune system has the greatest potential for the specific destruction of tumors with no toxicity to normal tissue and for long-term immunity that can prevent cancer recurrence in a long-lasting fashion. Yet, the efficacy of such agents may be improved.

Recently, it was shown that the composition of the microbiota has a major influence on the effectiveness of anticancer immunosurveillance and thereby may contribute to the therapeutic activity of immune-checkpoint inhibitors that target cytotoxic T lymphocyte protein 4 (CTLA-4) or the programmed cell death protein 1 (PD-1)/programmed cell death 1 ligand 1 (PD-L1) axis, as well as the activity of immunogenic chemotherapies (Routy et al., Nat Rev Clin Oncol. 2018 June; 15(6):382-396).

In a more general context, alteration of the microbiota has been associated with impaired chemotherapy efficacy. In particular, anti-Gram-positive antibiotics can have a negative impact on the anticancer activity of some chemotherapy agents, such as cyclophosphamide or cisplatin (Pflug, et al., Oncoimmunology, 2016 Apr. 22; 5(6):e1150399).

The disruption of the microbiota is often referred to as dysbiosis and can be characterized in terms of decrease in diversity and shift in composition of the microbiota. Among other molecules, antibiotics have recently been shown to profoundly disrupt the microbiota with disruptions lasting up to months after the antibiotic intake.

Therefore, it would be advantageous to provide solutions for preventing the disruption of the intestinal microbiota caused by the use of dysbiosis-inducing agents, such as antibiotics, in patients receiving or about to receive anticancer agents, in particular immuno-oncology agents, for improving their efficacy and, among other clinically relevant outcomes, increase tumour progression-free survival and overall survival of patients.

### SUMMARY OF THE INVENTION

The invention relates to a method for improving the efficacy of an anticancer agent in a subject in need thereof, comprising administering to said subject an effective amount of an adsorbent. The invention also relates to an adsorbent for use to improve the efficacy of an anticancer agent. The invention further relates to an adsorbent for use in a method for the treatment or prevention of a cancer, in combination with an anticancer agent, such as an immuno-oncology agent.

Thanks to the invention, the efficacy of an anticancer agent is improved. In particular, the efficacy of an immuno-oncology agent is improved. Without wishing to be bound to any theory, it is believed that this improvement is due to the preservation of the commensal microbiota of the gut, thereby preserving anticancer immunosurveillance and even reinforcing immune activity against cancer.

In a particular embodiment, the subject is a mammal subject, preferably a human subject.

In a further particular embodiment, the subject has received, receives, or will receive a dysbiosis-inducing pharmaceutical agent. In a particular embodiment, the dysbiosis-inducing pharmaceutical agent is an antibiotic administered to the subject for the prevention or the treatment of an infection. In this context, the adsorbent is administered to prevent the adverse effects the antibiotic may have on the commensal microbiota in the intestine, in particular in the lower part of the intestine, such as in the late ileum, the caecum or the colon.

In another embodiment, the subject does not receive an antibiotic treatment. In this context, the adsorbent is administered to prevent the disruption of the commensal microbiota of the gut for other reasons than for the administration of an antibiotic. For example, the adsorbent may be used to treat an infection from a harmful bacteria, such as from Clostridium difficile, by either directly impacting the growth of the harmful bacteria or by adsorbing toxins released by such harmful bacteria. In another example, the adsorbent may be used to mitigate the side effects of some treatments with pharmacological or other agents given to the patient that could have deleterious effects on the intestinal microbiota.

In a particular embodiment, the adsorbent is activated charcoal.

In another aspect, the subject may be administered with an antibiotic-inactivating enzyme instead of an adsorbent. In a further particular embodiment, the antibiotic-inactivating enzyme is a beta-lactamase. In a further particular embodiment, the antibiotic-inactivating enzyme is an erythromycin-esterase.

In a further particular embodiment, the adsorbent or the antibiotic-inactivating enzyme is for oral administration.

In another particular embodiment, the adsorbent or the antibiotic-inactivating enzyme is in a formulation that releases the adsorbent or antibiotic-inactivating enzyme in a desired part of the intestine, particularly in the lower part of the intestine, particularly in the late ileum, the caecum or the colon.

In some embodiments, the anticancer agent may be selected from, but is not limited to:
a tubulin poison, a taxane, e.g. docetaxel, paclitaxel,
a platinum compound, e.g. cisplatin, carboplatin, oxaliplatin,
an agent interfering with DNA replication such as DNA intercalating agents, e.g. anthracycline,
a topoisomerase inhibitor such as etoposide,
an antimetabolite, e.g. methotrexate, cytarabine (ara-C), gemcitabine, 5-Fluorouracil,
an alkylating agent e.g. mechlorethamine, melphalan, carmustine, ifosfamide, or cyclophosphamide,
a targeted agent, such as an enzyme inhibitor, in particular a kinase inhibitor, e.g. erlotinib, sorafenib, imatinib, or a proteasome inhibitor such as bortezomib, carfizomib, ixazomib,
a monoclonal antibody targeting the extracellular region of a growth factor receptor, such as trastuzumab, bevacizumab and cetuximab,
an immuno-oncology agent such as PD-1 or PD-L1 inhibitors e.g. pembrolizumab, nivolumab, durvalumab, atezolizumab, avelumab, durvalumab or drugs targeting CTLA-4 such as ipilimumab, and
a combination thereof, in particular combinations of chemotherapeutic agents and immuno-oncology agents.

In some embodiments, the immuno-oncology agent may be selected from, but without limitation:
an immune checkpoint inhibitor such as a PD-1 inhibitor, e.g. nivolumab or pembrolizumab,
a PDL-1 inhibitor, e.g. atezolizumab, avelumab, or durvalumab; or a CTLA-4 inhibitor, e.g. ipilimumab,
a cancer vaccine, e.g. sipuleucel-T,
an immunomodulator such as thalidomide, lenalidomide, pomalidomide,
a non-specific immunotherapy agent, e.g. interferons, or interleukins,
chimeric antigen receptor (CAR)-T cell therapy, e.g. tisagenlecleucel, or axicabtagene ciloleucel, and
combinations thereof.

### DETAILED DESCRIPTION OF THE INVENTION

Adsorbent and Adsorbent Formulations

The term “adsorbent” designates any compound or material that can adsorb a substrate, typically by physico-chemical binding between the adsorbent surface and the substrate(s) to be adsorbed. Adsorbents may be specific or non-specific. Preferred adsorbents for use in the invention are pharmaceutical grade adsorbents, best suited for use in humans or animals for pharmaceutical or veterinary applications.

Examples of adsorbents suitable for use in the present invention include, without limitation, activated charcoal (also referred to as activated carbon); clays, including bentonite, kaolin, montmorrillonite, attapulgite, halloysite, laponite, and the like; silica, including colloidal silica (Ludox® AS-40 for example), mesoporous silica (MCM41), fumed silica, zeolites and the like; talc; cholesteramine and the like; polystyrene sulfonates and the like; mono and polysulfonated resins; as well as other resins such as those used for bacteriologic testing such as BACTEC® resins.

Preferred adsorbents are activated charcoals (such as from Chemviron, Cabot, Norit, Jacobi Carbons, Merck Millipore, Sigma Aldrich, Desotec, Haycarb, Donau Carbon, or other sources) which are of pharmaceutical grade. In a particular embodiment, the adsorbent is activated charcoal, more particularly an activated charcoal having a specific surface area above 600 m²/g, in particular above 800 m²/g, in particular above 1000 m²/g, in particular above 1200 m²/g, in particular above 1400 m²/g, in particular above 1600 m²/g, even more particularly above 1800 m²/g. The activated charcoal may be of vegetal, mineral or synthetic origin, its surface being optionally modified by a physical or chemical treatment. In a particular embodiment, the activated charcoal is of vegetal origin. In a particular embodiment, the activated charcoal is derived from peat. In a particular embodiment, the activated charcoal is derived from coconut husks. In a particular embodiment, the activated charcoal is derived from different sources mixed together such as peat and coconut husks. In a particular embodiment, the activated charcoal is characterized by a European molasses number (of note the European molasses number is inversely related to the North American molasses number) which is preferably higher than 100, even more particularly greater than 200, even more particularly greater than 300, even more particularly greater than 400, even more particularly greater than 500, even more particularly greater than 600. In a particular embodiment, the activated charcoal has a phenazone number (measured according to the EU Pharmacopeia) greater than 10 g/100 g, even more particularly greater than 20 g/100 g, even more particularly greater than 30 g/100 g, even more particularly greater than 40 g/100 g, even more particularly greater than 50 g/100 g, even more particularly greater than 60 g/100 g. In a particular embodiment, the activated charcoal is characterized by a density between 0.05 and 0.8, even more particularly between 0.1 and 0.6, even more particularly between 0.15 and 0.5, even more particularly between 0.2 and 0.4.

The amount of adsorbent employed in the methods of the invention may vary depending upon the host/material being treated and the overall capacity, adsorption power and selectivity of the adsorbent. Typically, the amount of adsorbent is an amount sufficient to improve the efficacy of an anticancer agent. In a particular embodiment the amount of adsorbent is an amount sufficient to prevent the deleterious impact of a substance, such as an antibiotic, on the intestinal microbiota known as “dysbiosis” or disruption of the gut microbiota. In particular, the amount of adsorbent is an amount sufficient to improve the efficacy of an immuno-oncology agent, or to improve the effectiveness of anticancer immunosurveillance in a subject.

The adsorbent for use in the present invention may be formulated in a composition, such as a pharmaceutical composition, which may comprise pharmaceutically acceptable excipients, carriers, and/or additives. Such compositions include formulations for oral delivery, rectal delivery, local application, mucosal application, inhalation, and the like. In a particular embodiment, the adsorbent is formulated in a pharmaceutical composition suitable for administration to humans or animals. More preferably, the adsorbent is formulated in an oral formulation suitable to release said adsorbent in the intestine or in contact with intestinal bacteria, particularly in the gastrointestinal tract, more particularly in the lower part of the intestine, i.e. in the late ileum, the caecum and/or the colon.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Examples of formulations suitable for intestinal delivery of an adsorbent have been described in WO2006/122835 and WO2007/132022. In another embodiment, the adsorbent is formulated in a core. Preferably, the amount of adsorbent is between about 60% and about 100%, more preferably between about 70% and about 98%, more preferably between about 75% and about 95%, more preferably between about 80% and about 90% of the total weight of the core. In a preferred embodiment, the absorbent is formulated with a carrageenan, preferably in the form of a pellet, as proposed in WO2011/104275. Such a formulation can form a core. Such core may be covered with a layer of a coating such that the adsorbent is released in the lower part of the intestine, i.e., in the late ileum, caecum and/or colon. Alternatively, multiple cores may be included or embedded in a dosage unit form suitable for releasing its content in the lower part of the intestine, i.e. in the late ileum, caecum and/or colon, such as a capsule whose shell is suitable for releasing its content in the lower part of the intestine. In another embodiment, the pellets can be included in capsules themselves included in a coated capsule. In another embodiment, the pellets can be included or embedded in Multiple Unit Particle Systems.

Carrageenan is a naturally-occurring family of linear sulphated polysaccharides which are extracted from red seaweeds. Carrageenans are high molecular weight polysaccharides made up of repeating galactose and 3, 6-anhydrogalactose (3,6-AG) units, both sulfated and non-sulfated. The units are joined by alternating alpha 1-3 and beta 1-4 glycosidic linkages. Three basic types of carrageenan are available commercially, i.e. kappa, iota, and lambda carrageenans, which differ by the number and position of the ester sulfate groups on the galactose units. The carrageenan for use in the present invention can be selected from kappa, iota and lambda carrageenans, and mixtures thereof. In one aspect of this embodiment, the adsorbent is mixed with kappa-carrageenan. In a particular embodiment, the mixture comprises activated charcoal and kappa-carrageenan. Preferably, the amount of carrageenan is between about 5% and about 25%, more preferably between about 10% and about 20%, of the total weight of the adsorbent and the carrageenan. In a further particular embodiment, the amount of adsorbent (in particular activated charcoal) in the mixture is between about 95% and about 75%, more preferably between about 90% and about 80%, of the total weight of the adsorbent and the carrageenan. According to a specific embodiment of the invention, the amount of carrageenan is about 15% of the total weight of the adsorbent and the carrageenan. For example, the mixture may contain 85% of an adsorbent and 15% of carrageenan.

According to a particular embodiment of the invention, a mixture of activated charcoal and carrageenan, in particular kappa-carrageenan, is provided with the weight ratios indicated above.

The core (or pellet) may be produced by any suitable means known to the skilled artisan. In particular, granulation techniques are adapted to produce said core. For example, the core may be obtained by mixing the adsorbent and the carrageenan in the ratios indicated above, adding a solvent such as water to proceed to wet granulation, followed by extrusion, optionally followed by spheronization or pelletization with rotary knife, or one-pot pelletization. Any remaining water can be removed, for example, by drying the resulting pellets using conventional techniques.

In one embodiment, the core, or pellet has an average particle size in the range from 50 μm to 6000 μm, in particular 100 μm to 5000 μm, in particular 150 μm to 4000 μm, in particular 250 to 3000 μm, in particular 250 to 1000 μm, in particular 300 to 3000 μm (such as 500 to 3000 μm), in particular 300 to 1000 μm, in particular 500 to 1000 μm, in particular 500 to 700 μm.

The core composition can further include conventional excipients such as anti-adherents, binders, fillers, diluents, flavours, coloration agents, lubricants, glidants, preservatives, sorbents and/or sweeteners. The amounts of such excipients can vary, but are typically in the range of 0.1 to 50% by weight of the pellet.

As discussed above, a preferred formulation of the invention comprises a core comprising an adsorbent, possibly supplemented with carrageenan, which core is covered with a layer of a coating such that the adsorbent is released in the lower part of the intestine, i.e., in the late ileum, caecum and/or colon.

In this regard, in a preferred embodiment, the adsorbent is used as a formulation comprising:
a core containing the adsorbent, and
a layer of an external coating formed around the core such that the adsorbent is released from the formulation in the lower part of the intestine.

In a preferred embodiment, the adsorbent is used as a formulation comprising:
a core containing the adsorbent and carrageenan, and
a layer of an external coating formed around the core such that the adsorbent is released from the formulation in the lower part of the intestine.

Examples of suitable coatings include pH-dependent enterosoluble polymers, azopolymers, disulphide polymers, and polysaccharides, in particular amylose, pectin (e.g. pectin crosslinked with divalent cations such as calcium pectinate or zinc pectinate), chondroitin sulphate and guar gum. Representative pH-dependent enterosoluble polymers include cellulose acetate trimellitate (CAT), cellulose acetate phthalate (CAP), acrylic polymers, methacrylic polymers, anionic copolymers based on methylacrylate, methylmethacrylate and methacrylic acid, hydroxypropyl methylcellulose phthalate (HPMCP), hydroxypropylmethylcellulose acetate succinate (HPMCAS), methacrylic acid and ethyl acrylate copolymers, methacrylic acid and methyl methacrylate copolymers in a 1:1 molar ratio, methacrylic acid and methyl methacrylate copolymers in a 1:2 molar ratio, polyvinyl acetate phthalate (PVAP) and shellac resins. Particularly preferred polymers include shellac, anionic copolymers based on methyl acrylate, methyl methacrylate and methacrylic acid, such as poly(methyl acrylate-co-methyl methacrylate-co-methacrylic acid) in a 7:3:1 molar ratio, as well as methacrylic acid and methyl methacrylate copolymers in a 1:2 molar ratio. Ideally, the polymer dissolves at a pH equal to 6.0 and above, preferably 6.5 and above. Suitable coatings may also be obtained by mixing the polymers and copolymers aforementioned. In another embodiment, suitable coatings are time-dependent coatings or based on time-dependent polymers such as mixture of ethylcellulose polymers with alginate sodiums.

In a particular embodiment, the formulation comprises a further intermediate coating located between the core and the external pH-dependent layer. The intermediate coating can be formed from a variety of polymers, including pH-dependent polymers, pH-independent water soluble polymers, pH-independent insoluble polymers, and mixtures thereof. Examples of such pH-dependent polymers include shellac type polymers, anionic copolymers based on methylacrylate, methylmethacrylate and methacrylic acid, methacrylic acid and ethyl acrylate copolymers, hydroxypropyl methylcellulose phthalate (HPMCP), and hydroxypropylmethylcellulose acetate succinate (HPMCAS). Examples of pH-independent water soluble polymers include PVP or high molecular weight cellulose polymers such as hydroxypropylmethylcellulose (HPMC) or hydroxypropylcellulose (HPC). Examples of pH-independent insoluble polymers include ethylcellulose polymers or ethyl acrylate and methyl methacrylate copolymers.

In a particular embodiment, the invention uses a formulation comprising:
a core comprising a mixture of an adsorbent (preferably activated charcoal) with carrageenan (preferably kappa-carrageenan),
an intermediate coating selected in the group consisting of HPMC, ethylcellulose and a mixture of methacrylic acid and ethyl acrylate copolymer such as Eudragit® L30D-55, and ethyl acrylate and methyl methacrylate copolymer such as Eudragit® NE30D (for example in a mixture weight ratio of 1:9 to 9:1, preferably of 2:8 to 3:7), and
an external layer of an anionic copolymer based on methyl acrylate, methyl methacrylate and methacrylic acid, such as poly(methyl acrylate-co-methyl methacrylate-co-methacrylic acid) 7:3:1, e.g. Eudragit® FS30D.

In a specific embodiment, the formulation comprises a core, comprising about 85% activated charcoal and about 15% kappa-carrageenan, and a coating with an anionic copolymer based on methyl acrylate, methyl methacrylate and methacrylic acid (such as poly(methyl acrylate-co-methyl methacrylate-co-methacrylic acid) 7:3:1, e.g. Eudragit® FS30D, Evonik, Darmstadt, Germany) or a mixture of methacrylic acid and ethyl acrylate copolymer (such as Eudragit® L30D55, Evonik, Darmstadt, Germany).

In another embodiment, the adsorbent is formulated in a composition as disclosed in WO2014044794, comprising:
(a) a core comprising activated carbon;
(b) a first layer around the core, the first layer comprising an insoluble semipermeable material; and
(c) a second layer around the first layer which dissolves at a predetermined pH or which dissolves at a predetermined location in the gastrointestinal tract.

In a variant of this embodiment, the core is activated carbon. In another variant, the activated carbon is sanded or deburred. In yet a further variant, the activated carbon is of particle size 0.02 to 5.0 mm, for example of particle size 0.6 to 1.2 mm. In a further variant, the insoluble semipermeable material comprises one or more of ethyl cellulose, glycerylmonostearate, cellulose acetate butyrate, dipolylactic acid, polyvinyl chloride, and a poly(meth)acrylate polymer such as Eudragit RL 100, Eudragit RL PO, Eudragit RL 30D, Eudragit RL 12.5, Eudragit RS 100, Eudragit RS PO, Eudragit RS 30D, Eudragit RS 12.5 and Eudragit NE 30D, Eudragit HE 40D. In another variant, the first layer further comprises a water soluble material, wherein the first layer may further comprise a water soluble material comprising hydroxypropylmethyl cellulose (HPMC). Said water soluble material may be mixed with the insoluble semipermeable material in certain embodiments and/or may comprise 0.1 to 30% by weight of the amount of the insoluble semipermeable material, for example 2 to 25% by weight of the amount of the insoluble semipermeable material. In a further particular variant, the first layer allows gradual diffusion of molecules through the semipermeable membrane towards the core into contact with the activated carbon. In yet another variant, the second layer comprises a material which dissolves at pH 5 to pH 7. In some variants, the second layer is an enteric layer comprising a material which remains substantially intact at pH 1 to 4.9, but which breaks down rapidly at pH 5 to 7. In a variant, the second layer comprises a pH sensitive polymer. Representative second layers include layers selected from Hypromellose-Acetate-Succinate, cellulose acetate trimellitate (CAT), cellulose acetate phthalate (CAP), anionic copolymers based on methylacrylate, methylmethacrylate and methacrylic acid, hydroxypropyl methylcellulose phthalate (HP CP), hydroxypropylmethylcellulose acetate succinate (HPMCAS), methacrylic acid and ethyl acrylate copolymers, methacrylic acid and ethyl acrylate copolymers, methacrylic acid and methyl methacrylate copolymers (1:1 molar ratio), methacrylic acid and methyl methacrylate copolymers (1:2 molar ratio), Polyvinyl acetate phthalate (PVAP) and Shellac resins. In a further particular variant of this embodiment, the activated carbon is the sole active pharmaceutical ingredient. In still another variant, the composition comprises:

(a) a core comprising activated carbon;

(b) a first layer around the core, the first layer comprising an insoluble semipermeable material in the form of ethyl cellulose, and optionally further comprising a water soluble material comprising hydroxypropylmethylcellulose (HPMC); and

(c) a second layer comprising hydroxypropylmethylcellulose acetate succinate (HPMC AS).

In another variant, the adsorbent is activated carbon formulated in a composition comprising:

(a) a core which is activated carbon;

(b) a first layer around the core, the first layer comprising a semipermeable material which is insoluble in water and further comprises a water soluble material comprising hydroxypropylmethyl cellulose in an amount of 2-25% by weight of the amount of the insoluble semipermeable material; and

(c) a second layer around the first layer which dissolves at pH 5 to 7.

Antibiotics

The term “antibiotic” designates any compound that is active against bacteria. Antibiotics that may be eliminated thanks to the invention include but are not limited to:
beta-lactams including:
   penicillins (such as penicillin G, penicillin V, ampicillin, amoxicillin, bacampicillin, carbenicillin, carbenicillin indanyl, ticarcillin, azlocillin, mezlocillin, piperacillin, and the like),
   penicillinase-resistant penicillins (such as methicillin, oxacillin, cloxacillin, dicloxacillin, nafcillin and the like),
   cephalosporins, such as: first generation cephalosporins (such as cefadroxil, cephalexin, cephradine, cephalothin, cephapirin, cefazolin, and the like); second generation cephalosporins (such as cefaclor, cefamandole, cefonicid, cefoxitin, cefotetan, cefuroxime, cefuroxime axetil, cefinetazole, cefprozil, loracarbef, ceforanide, and the like); third generation cephalosporins (such as cefepime, cefoperazone, cefotaxime, ceftizoxime, ceftriaxone, ceftazidime, cefixime, cefpodoxime, ceftibuten, and the like); fourth generation cephalosporins (such as cefclidine, cefepime, cefozopran, cefpirome, cefquionome and the like); fifth and further generation cephalosporins (such as ceftobiprole, ceftaroline, ceftolozane and the like),
   carbapenems (such as imipenem, meropenem, ertapenem, doripenem and the like)
   monobactams (such as aztreonam, and the like),
quinolones (such as nalidixic acid) and fluoroquinolones (such as cinoxacin, ciprofloxacin, moxifloxacin, levofloxacin, ofloxacin, gatifloxacin, gelifloxacin, norfloxacin and the like),
sulfonamides (e.g., sulfanilamide, sulfadiazine, sulfamethoxazole, sulfisoxazole, sulfacetamide, sulfamethoxydiazine and the like),
aminoglycosides (e.g., streptomycin, gentamicin, tobramycin, amikacin, netilmicin, kanamycin, neomycins B, C and E), spectinomycin, puromycin, gentamicin, and the like),
tetracyclines (such as tetracycline, chlortetracycline, oxytetracycline, methacycline, doxycycline, minocycline, tigecycline, eravacycline and the like),
macrolides (such as erythromycin, azithromycin, clarithromycin, fidaxomicin, telithromycin, josamycin, oleandomycin, spiramycin, tylosin, roxithromycin, cethromycin, solithromycin, and the like),
glycopeptides (such as vancomycin, oritavancin, telavancin, teicoplanin, dalbavancin, ramoplanin and the like),
oxazolidinones (such as linezolid, posizolid, tedizolid, radezolid, cycloserine and the like),
phenicols (such a chloramphenicol, tiamphenicol and the like),
lincosamides (such as clindamycin, lincomycin and the like),
Streptogramins (such as pristinamycin, quinupristin/dalfopristin, virginiamycin and the like)
polymyxins (such as polymyxin A, B, C, D, E1 (colistin A), or E2, colistin B or C, and the like),
diaminopyrimidines (such as trimethoprim, often used in conjunction with sulfamethoxazole, pyrazinamide, and the like),
sulfones (such as dapsone, sulfoxone sodium, and the like),
para-aminobenzoic acid,
bacitracin,
isoniazid,
rifamycins (such as rifampicin, rifabutin, rifapentine, rifalasil, rimamixin, and the like)
ethambutol,
ethionamide,
capreomycin,
clofazimine, and
any other antibacterial agent.

The term “antibiotic” also covers combinations of antibiotics.

Antibiotic-Inactivating Enzymes and Enzyme Formulations

In certain embodiments, the invention implements antibiotic-inactivating enzymes to improve the efficacy of an anticancer agent in a subject in need thereof, wherein the subject has received, receives, or will receive an antibiotic for the prevention or the treatment of an infection.

In the context of the present invention, an “antibiotic-inactivating enzyme” is an enzyme able to hydrolyse or inactivate an antibiotic, thereby rendering said antibiotic biologically inactive. For example, an antibiotic-inactivating enzyme may substantially increase the minimal inhibitory concentration (MIC) of an antibiotic in comparison to the MIC obtained without said enzyme. According to the present invention, an antibiotic inactivation is total if growth of bacteria, sensitive to a certain concentration of a given antibiotic, in the presence of said concentration of the antibiotic after its treatment with the inactivating enzyme, is identical to growth in the absence of the antibiotic. Another definition of total inactivation is when the MIC of an antibiotic for sensitive bacteria is increased by at least 2 orders of magnitude after treatment with the inactivating enzyme.

Antibiotic-inactivating enzymes for use according to the invention can be natural, chemically modified, genetically engineered or synthetic.

Antibiotic-inactivating enzymes also include functional variants of a parent antibiotic-inactivating enzyme, such as functional variants of a beta-lactamase, erythromycin esterases and ketoreductases. In the context of the present invention, a “functional variant” of an enzyme is an enzyme deriving from a parent enzyme, that has the same type of catalytic activity (for example, a beta-lactamase variant is an enzyme that has beta-lactamase activity), but with a different amino acid sequence. Such a functional variant may have at least 70%, 75%, 80%, 85%, 90%, 91%, 92%, 93%, 94%, 95%, 96%, 97%, 98%, 98.5%, 99%, 99.1%, 99.2%, 99.3%, 99.4%, 99.5%, 99.6%, 99.7%, 99.8%, at least 99.9% identity to the parent enzyme. Such a functional variant may also have a specific activity for a given antibiotic, such as for a given beta-lactam antibiotic in case of a beta-lactamase, of a least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 220%, 240%, 260%, 280%, 300%, 350%, 400%, 500%, 600%, 700%, 800% or even at least 1600%, relative to the specific activity of the parent antibiotic-inactivating enzyme.

Representative antibiotic-inactivating enzymes that may be used in the practice of the present invention include, without limitation, an enzyme inactivating a beta-lactam antibiotic (such as beta-lactamases), an enzyme inactivating a fluoroquinolone (such as aminoglycoside N-acetyltransferases), an enzyme inactivating a macrolide (such as erythromycin-esterases or erythromycin-phosphotransferases), an enzyme inactivating a tetracycline (such as NADPH-dependent oxydoreductase-tetracyclines) or an enzyme inactivating a lincosamide (such as nucleotidyltransferase-lincomycines).

A beta-lactamase is an enzyme (EC 3.5.2.6) having beta-lactamase activity, i.e. an enzyme which catalyzes the irreversible hydrolysis of the amide bond of the beta-lactam ring found in compounds such as beta-lactam antibiotics (e. g. penicillins, cephalosporins, carbapenems, penam sulfones) to create an hydrolyzed molecule devoid of its antibacterial activity. This class of enzymes is well known to those skilled in the art (Wang et al., 1999, Curr Opin Chem Biol. 3(5),614-22; Frere, J. M. 1995, Mol Microbiol. 16(3):385-95).

In a particular embodiment, the beta-lactamase is a serine beta-lactamase or a zinc-dependent beta-lactamase, also referred to as metallo-beta-lactamase. In another embodiment, the beta-lactamase is selected from class A, class B, class C and class D beta-lactamases. In a further particular embodiment, the beta-lactamase is selected from group 1, group 2, group 3 and group 4 beta-lactamases (Bush et al., Antimicrob. Agents Chemother, 39: 1211). In some embodiments, the beta-lactamase is one or more of P1A, P3A or P4A and their derivatives which consist in derivatives of the beta-lactamase from Bacillus lichenoformis 749/C, or P2A which is the metallo beta-lactamase from Bacillus cereus and derivatives thereof. Furthermore, the beta-lactamase may be an extended-spectrum beta-lactamase (ESBL), optionally selected from a TEM, SHV, CTX-M, OXA, PER, VEB, GES, and IBC beta-lactamase. Further, the beta-lactamase may be an inhibitor-resistant β-lactamase, optionally selected from an AmpC-type β-lactamases, a carbapenemase such as, but not limited toi IMP-type carbapenemases (metallo-β-lactamases), VIM (Verona integron-encoded metallo-β-lactamase) carbapenemases, OXA (oxacillinase) group of β-lactamases, KPC (K. pneumonia carbapenemase), CMY (Class C), SME, IMI, NMC and CcrA, and a NDM (New Delhi metallo-β-lactamase, e.g. NDM-1) beta-lactamases.

In some embodiments, the beta-lactamase is a VIM (Verona integron-encoded metallo-beta-lactamase). Illustrative VIM enzymes include, but are not limited to, VIM-1, VIM-2, VIM-3, VIM-4, and VIM-19. Additional VIM enzymes are described in, for example, Queenan of al. (2007) Clin. Microbiol. Rev. 20(3):440-458. In a further particular embodiment, the beta-lactamase is VIM-2 or a variant thereof. Such beta-lactamases are disclosed in PCT/EP2017/053985, PCT/EP2017/053986 and EP17198414. In specific aspects, the present invention relates to the use of any specific embodiment disclosed in PCT/EP2017/053985, PCT/EP2017/053986 and EP17198414, including any specific variant VIM-2 disclosed therein. In a particular embodiment, the antibiotic-inactivating enzyme is VIM-2, such as represented in SEQ ID NO:1. In another particular embodiment, the antibiotic-inactivating enzyme is a VIM-2 functional variant having an amino acid sequence as shown in SEQ ID NO:2 to 46. In a particular embodiment, the VIM-2 functional variant has a sequences comprising or consisting of SEQ ID NO:29; SEQ ID NO:31, SEQ ID NO:34 or SEQ ID NO: 36.

In another embodiment, the beta-lactamase is the beta-lactamase from Bacillus lichenoformis 749/C or a variant thereof, such as P1A, P3A (also referred to as “ribaxamase”) or P4A. P1A has the sequence shown in SEQ ID NO:47.

In some embodiments, the beta-lactamase is the metallo beta-lactamase from Bacillus cereus (also known as P2A), or a functional variant thereof, as described, for example, in WO2007147945. In a particular embodiment, the P2A enzyme has the sequence shown in SEQ ID NO:48. A functional variant of the P2A enzyme may have at least about 60%, at least about 70%, at least about 80%, at least about 90%, at least about 95%, at least about 97%, at least about 98%, at least about 99%, or 100% identity to sequence shown in SEQ ID NO:48.

In some embodiments, the beta-lactamase is P3A or a functional variant thereof, as described, for example, in WO2011148041. In a particular embodiment, the P3A enzyme has the sequence shown in SEQ ID NO:49 (mature form of the enzyme) or SEQ ID NO:50 (form of the enzyme including a 31 amino acid long signal peptide). A functional variant of the P3A enzyme may have at least about 60%, at least about 70%, at least about 80%, at least about 90%, at least about 95%, at least about 97%, at least about 98%, at least about 99%, or 100% identity to sequence shown in SEQ ID NO:49 or SEQ ID NO:50. In a particular embodiment, the beta-lactamase comprises an amino acid sequence having at least 80% sequence identity with SEQ ID NO:49, and is characterized in that it has a hydrophilic amino acid residue other than aspartic acid (D) at a position corresponding to position 276 according to Ambler classification and said hydrophilic amino acid is selected from arginine (R), histidine (H), lysine (K), asparagine (N), glutamine (Q), serine (S) and threonine (T). In a further particular embodiment, the beta-lactamase comprises an amino acid sequence having at least 80% sequence identity with SEQ ID NO:49, and is characterized in that it has an asparagine (N) at a position corresponding to position 276 according to Ambler classification. In yet another embodiment, the beta-lactamase has the amino acid sequence shown in SEQ ID NO:49, wherein the amino acid residue at the position corresponding to position 276 according to Ambler classification is an asparagine (N).

in another embodiment, the beta-lactamase is P4A or a functional variant thereof, as described, for example, in WO2015/161243. In a particular embodiment, the P4A enzyme has the sequence of SEQ ID NO:79 or SEQ ID NO:80. A functional variant of the P4A enzyme may have at least about 60%, at least about 70%, at least about 80%, at least about 90%, at least about 95%, at least about 97%, at least about 98%, at least about 99%, or 100% identity to sequence shown in SEQ ID NO:79 or SEQ ID NO:80.

In some embodiments, the beta-lactamase is a Klebsiella pneumoniae carbapenemase (KPC). Illustrative KPCs include, but are not limited to, KPC-1/2 (SEQ ID NO:51), KPC-3 (SEQ ID NO:52), KPC-4 (SEQ ID NO:53), KPC-5 (SEQ ID NO:54), KPC-6 (SEQ ID NO:55), KPC-7 (SEQ ID NO:56), KPC-8 (SEQ ID NO:57), KPC-9 (SEQ ID NO:58), KPC-10 (SEQ ID NO:59), KPC-11 (SEQ ID NO:60), KPC-12 (SEQ ID NO:61), KPC-13 (SEQ ID NO:62), KPC-14 (SEQ ID NO:63), KPC-15 (SEQ ID NO:64), and KPC-17 (SEQ ID NO:65). In an embodiment, the beta-lactamase is KPC-1/2. In an embodiment, the beta-lactamase is KPC-3. The functional variants of KPC enzymes may have at least about 60%, at least about 70%, at least about 80%, at least about 90%, at least about 95%, at least about 97%, at least about 98%, at least about 99%, or 100% identity to the sequences shown in SEQ ID NO:51 to SEQ ID NO:65.

In another embodiment, the beta-lactamase is a New Delhi metallo-beta-lactamase (NDM). Illustrative NDMs include, without limitation, NDM-1 (SEQ ID NO:66), NDM-2 (SEQ ID NO:67), NDM-3 (SEQ ID NO:68), NDM-4 (SEQ ID NO:69), NDM-5 (SEQ ID NO:70), NDM-6 (SEQ ID NO:71), NDM-7 (SEQ ID NO:72), NDM-8 (SEQ ID NO:73), NDM-9 (SEQ ID NO:74), NDM-10 (SEQ ID NO:75), NDM-11 (SEQ ID NO:76), NDM-12 (SEQ ID NO:77), and NDM-13 (SEQ ID NO:78). In an embodiment, the beta-lactamase is NDM-1. In an embodiment, the broad spectrum carbapenemase is NDM-4. The functional variants of NDM enzymes may have at least about 60%, at least about 70%, at least about 80%, at least about 90%, at least about 95%, at least about 97%, at least about 98%, at least about 99%, or 100% identity to sequences shown in SEQ ID NO:66 to SEQ ID NO:78.

In some embodiments, the beta-lactamase is an IMP-type carbapenemase. Illustrative IMP-type enzymes include, without limitation, IMP-1, IMP-4, IMP-8, IMP-11, IMP-43 and IMP-44. Additional IMP-type enzymes are described in, for example, Queenan of al. (2007) Clin. Microbiol. Rev. 20(3):440-458.

In some embodiments, the beta-lactamase from the OXA (oxacillinase) group of beta-lactamases. Illustrative OXA beta-lactamases include, without limitation, OXA-23, OXA-24, OXA-27, OXA-40, OXA-48, OXA-49, OXA-50, OXA-51, OXA-58, OXA-64, OXA-71, and OXA-181. Additional OXA type beta-lactamases are described in, for example, Walther-Rasmussen et al., Journal of Antimicrobial Chemotherapy (2006), 57:373-383 and Queenan et al. (2007) Clin. Microbiol. Rev. 20(3):440-458.

In some embodiments, the beta-lactamase is a CMY (class C carbapenemase) enzyme. An illustrative CMY enzyme with carbapenemase activity is CMY-10, as described in, for example, Lee et al., (2006) Research Journal of Microbiology (1): 1-22.

In some embodiments, the beta-lactamase is a SME enzyme (for Seiratia marcescens). Illustrative SME enzymes include, without limitation, SME-1, SME-2 or SME-3, as described in, for example, Queenan et al. (2007) Clin. Microbiol. Rev. 20(3):440-458.

In some embodiments, the beta-lactamase is an IMI enzyme (imipenem hydrolyzing beta-lactamase). Illustrative IMI enzymes include, without limitation, IMI-1 or IMI-2, as described in, for example, Queenan et al. (2007) Clin. Microbiol. Rev. 20(3):440-458.

In some embodiments, the beta-lactamase is a NMC enzyme (not metalloenzyme carbapenemase). An illustrative NMC enzyme is NMC-A, as described in, for example, Queenan et al. (2007) Clin. Microbiol. Rev. 20(3):440-458.

In some embodiments, the beta-lactamase is a GES enzyme (Guiana extended spectrum). Illustrative GES enzymes include, without limitation, GE-2, GES-4, GES-5, GES-6, GES-7, GES-8, GES-9, GES-11, GES-14 and GES-18 as described in, for example, Queenan of al. (2007) Clin. Microbiol. Rev. 20(3):440-458 and Johnson et al., (2014) Crystal Structures of Class A, B, and D β-Lactamases (http://www.carbapenemase.ca/crystal_structures.html).

In some embodiments, the beta-lactamase is the CcrA (CfiA) metallo-beta-lactamase from Bacteroides fragilis.

In some embodiments, the beta-lactamase is the SFC-1 enzyme from Serratia fonticola or SHV-38 enzyme from Klebsiella pneumoniae, as described in, for example, Walther-Rasmussen et al., (2007) Journal of Antimicrobial Chemotherapy, 60:470-482.

In another embodiment, the antibiotic-inactivating enzyme is an erythromycin esterase. Erythromycin-esterase (EC number 3.1.1) refers to a class of enzymes that catalyze the inactivation of erythromycin as well as other macrolide antibiotics. These enzymes hydrolyze the lactone ring of macrolides such as erythromycin and oleandomycin as explained in Barthelemy et al. 1984, J. Antibiot. 37, 1692-1696. Known erythromycin-esterases are of bacterial origins. They are produced for example by Escherichia coli, Halobacterium salinarum, Gramella forsetii, Achromobacter denitrificans or Rhodococcus sp. In a particular embodiment, the erythromycin-esterase is one of the enzymes usually produced by members of the family Enterobacteriaceae highly resistant to erythromycin as described in Arthur et al. 1987, Antimicrob. Agents Chemother. 31(3), 404-409. Two erythromycin-esterases from E. coli have been documented under the reference names EreA and EreB, the use of both of which being envisioned in the present invention. In a particular embodiment of the invention, the erythromycin-esterase is the EreB erythromycin-esterase from E. coli (cf. Arthur et al. 1986, Nucleic Acids Res 14(12), 4987-4999).

In another embodiment, the antibiotic-inactivating enzyme is a ketoreductase. Ketoreductase (KRED) or carbonyl reductase class (EC 1.1.1.184) enzymes are useful for the synthesis of optically active alcohols from the corresponding prochiral ketone substrate. KREDs typically convert a ketone substrate to the corresponding alcohol product, but may also catalyze the reverse reaction, oxidation of an alcohol substrate to the corresponding ketone/aldehyde product.

In another embodiment, the antibiotic-inactivating enzyme is a hybrid protein molecule. Representative hybrid protein molecules are those disclosed in US Patent Application 20170354706. Such hybrid protein molecule may comprise two enzymes bonded together, capable of inactivating at least one antibiotic. In a particular embodiment, theses enzymes are combined into a single monocatenary protein. These two enzymes can be both from the same class, or each from different classes. For example, the two enzymes can be beta-lactamases, or chosen among the categories of beta-lactamases, enzymes inactivating an aminoglycoside, enzymes inactivating a fluoroquinolone, enzymes inactivating a lincosamide, enzymes inactivating a macrolide, or enzymes inactivating a tetracycline. In a particular embodiment, each enzyme in the hybrid protein molecule inactivates different antibiotics. In another embodiment, the hybrid protein molecule comprises two enzymes capable of inactivating antibiotics belonging to the same class. In a particular embodiment, the sequence of at least one of the component enzymes in the hybrid protein has a sequence homology of at least 40%, 45%, 50%, 55%, 60%, 65%, 70%, 75%, 80%, 85%, 90%, 91%, 92%, 93%, 94%, 95%, 96%, 97%, 98%, 98.5%, 99%, 99.1%, 99.2%, 99.3%, 99.4%, 99.5%, 99.6%, 99.7%, 99.8%, or at least 99.9% with SEQ ID NO:81 to SEQ ID NO:87. In further particular embodiment, the sequence of at least one of the component enzymes in the hybrid protein has a sequence consisting of SEQ ID NO:81, SEQ ID NO:82, SEQ ID NO:83, SEQ ID NO:84, SEQ ID NO:85, SEQ ID NO:86 or SEQ ID NO:87. In a further particular embodiment, the hybrid protein molecule has an amino acid sequence comprising or consisting of a sequence selected in the group consisting of SEQ ID NO:88 to 90.

In another embodiment, the enzyme, whether produced biologically or synthetically, may be further enzymatically and/or chemically modified in order to enhance its activity, stability, solubility or any other beneficial characteristics. One example of such modifications is the linking of polyethylene glycol, or PEGylation, to surface amino groups.

In a particular embodiment, the antibiotic-inactivating enzyme is formulated in a formulation suitable to release the enzyme in a desired part of the intestine. In a particular embodiment, the desired part of the intestine is the lower part of the intestine, such as the ileum, the caecum or the colon. In another particular embodiment, the desired part of the intestine is the upper part of the intestine, such as the duodenum or the jejunum. In a particular embodiment, the formulation comprises pellets of enzymes coated with an enteric coating (such as with an enteric coating dissolving at a pH greater or equal to 7.0). In another particular embodiment, the formulation comprises enteric-coated enzyme pellets (such as with an enteric coating dissolving at a pH greater or equal to 5.5 or at a pH greater or equal to 7.0) within enteric-coated capsules (such as with an enteric coating dissolving at a pH greater or equal to 5.5 or at a pH greater or equal to 7.0). In another particular embodiment, the formulation comprises enteric-coated pellets in uncoated capsules. The choice of the formulation may depend on the route of administration of the antibiotic to the subject. For example, in case of parenteral administration of the antibiotic, a formulation releasing the antibiotic-inactivating enzyme at the upper or lower part of the intestine may be considered. In case of oral administration the antibiotic, the enzyme formulation preferably releases the enzyme in the lower part of the intestine, at a location where the inactivation of the antibiotic by the enzyme cannot interfere anymore with the desired absorption of said antibiotic in the small intestine, in order to benefit from the positive effect of the antibiotic.

In a particular embodiment, the formulation includes inhibitors of digestive proteases such as pepsin, trypsin, chymotrypsin, carboxypeptidase, elastase, in order to preserve the enzyme and extend the time during which it is active in the gut.

Methods of Use

The present invention relates to an adsorbent as provided above, for use in a method for improving the therapeutic efficacy of an anticancer agent, such as an immuno-oncology agent. The invention also relates to an adsorbent as provided above, for use in a method for treating or preventing cancer, in combination with an anticancer agent, such as an immuno-oncology agent. The invention further relates to an adsorbent as provided above, for use in a method for treating or preventing cancer, in combination with an anticancer agent, such as an immuno-oncology agent, thereby improving the efficacy of said anticancer agent. The invention also relates to an adsorbent as provided above, for use in a method for treating or preventing cancer, in combination with an anticancer agent, such as an immuno-oncology agent, thereby preserving the efficacy of said anticancer agent. The invention further relates to an adsorbent as provided above, for use in a method for treating or preventing cancer, in combination with an anticancer agent, such as an immuno-oncology agent, thereby potentiating the efficacy of said anticancer agent.

The adsorbent may be administered at any point in the therapy, e.g. before, during and/or after the anticancer agent, such as an immuno-oncology agent. In particular, the adsorbent may be administered as soon as the patient is diagnosed with a malignancy, even if the intent to administer an anticancer agent only constitutes a remote possibility. Anticancer agents, also sometimes referred to as antineoplastic agents, are substances that act against cancer in a mammal, such as a human being. The term “anticancer agent” includes, without limitation, chemicals and biological agents that affect directly a cancer cell, or indirectly such as by affecting the vascularisation of the cancer cell. For example, anticancer agents include, without limitation, chemotherapeutic molecules such as cytostatic agents, cytotoxic agents and anti-angiogenesis agents, anticancer antibodies targeting cancer cells, anticancer peptides and anticancer viruses. Illustrative anticancer agents include, without limitation:
tubulin poisons, taxanes, e.g. docetaxel, paclitaxel,
platinum compounds, e.g. cisplatin, carboplatin, oxaliplatin,
agents interfering with DNA replication such as DNA intercalating agents, for example anthracyclines,
topoisomerase inhibitors such as etoposide,
antimetabolites, e.g. methotrexate, cytarabine (ara-C), gemcitabine, 5-Fluorouracil,
alkylators, e.g. mechlorethamine, melphalan, carmustine, ifosfamide, or cyclophosphamide,
targeted agents, such as enzyme inhibitor, in particular kinase inhibitors, e.g. erlotinib, sorafenib, imatinib, or proteasome inhibitors such as bortezomib, Carfizomib, Ixazomib,
monoclonal antibodies targeting the extracellular region of a growth factor receptor, such as trastuzumab, bevacizumab and cetuximab,
immuno-oncology agents, and
combinations thereof.

Anthracyclines include, without limitation, doxorubicin and daunorubicin.

Topoisomerase inhibitors further include, without limitation, camptothecin, irinotecan, topotecan, and derivatives thereof.

Antimetabolites further include, without limitation, capecitabine and pemetrexed.

In a particular embodiment, the anticancer agent is an immuno-oncology agent. Immuno-oncology agents (also known as immuno-targeted agents) act against tumors, at least in part, by involving the immune system, or by an immune system-related mode of action. An immuno-oncology may more particularly act by modulating the action of immune cells.

Examples of immuno-oncology agents comprise agents that modulate immune checkpoints such as 2B4, 4-1BB (CD137), AaR, B7-H3, B7-H4, BAFFR, BTLA, CD2, CD7, CD27, CD28, CD30, CD40, CD80, CD83 ligand, CD86, CD160, CD200, CDS, CEACAM, CTLA-4, GITR, HVEM, ICAM-1, KIR, LAG-3, LAIR1, LFA-1 (CD 11 a/CD 18), LIGHT, NKG2C, NKp80, OX40, PD-1, PD-L1, PD-L2, SLAMF7, TGFRp, TIGIT, Tim3 and VISTA.

Immuno-oncology agents may be in the form of antibodies, peptides, small molecules or viruses. In a particular embodiment, the immuno-oncology agent is an antibody against PD-1, PD-L1 or PD-L2.

In a particular embodiment, the immuno-oncology agent is an inhibitor of arginase, CTLA-4, indoleamine 2,3-dioxygenase, and/or PD-1/PD-L1. In certain embodiments, the immuno-oncology agent is abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab, ozogamicin, intelumumab, ipilimumab, isatuximab, lambrolizumab, MED 14736, MPDL3280A, nivolumab, obinutuzumab, ocaratuzumab, ofatumumab, olatatumab, pembrolizumab, pidilizumab, rituximab, ticilimumab, samalizumab, or tremelimumab.

More generally, an immuno-oncology agent may be any agent that may be used in the treatment of malignant diseases and that acts, at least in part, by involving the immune system, or has an immune system-related mode of action. For example, the immuno-oncology agent may be selected from, without limitation:
an immune checkpoint inhibitor such as a PD-1 inhibitor, e.g. nivolumab or pembrolizumab;
an immune checkpoint inhibitor such as a PDL-1 inhibitor, e.g. atezolizumab, avelumab, or durvalumab; or a CTLA-4 inhibitor, e.g. ipilimumab,
a cancer vaccine, e.g. sipuleucel-T;
an immunomodulator such as thalidomide, lenalidomide, pomalidomide,
a non-specific immunotherapy, e.g. interferons, or interleukins; and
a chimeric antigen receptor (CAR)-T cell therapy, e.g. tisagenlecleucel, or axicabtagene ciloleucel, and
combinations thereof.

In a particular embodiment, the anticancer agent is an anti-PD-1 antibody. In a further particular embodiment, the anti-PD-1 antibody is selected from nivolumab and pembrolizumab.

In a particular embodiment of the invention, the anticancer agent is selected from Afatinib, Aflibercept, Alemtuzumab, Alitretinoin, Altretamine, Anagrelide, Arsenic trioxide, Asparaginase, Atezolizumab, Avelumab, Axitinib, Azacitidine, Bendamustine, Bevacizumab, Bexarotene, Bleomycin, Bortezomib, Bosutinib, Busulfan, Cabazitaxel, Capecitabine, Carboplatin, Carmofur, Carmustine, Cetuximab, Chlorambucil, Chlormethine, Cisplatin, Cladribine, Clofarabine, Crizotinib, Cyclophosphamide, Cytarabine, Dacarbazine, Dactinomycin, Dasatinib, Daunorubicin, Decitabine, Denileukin diftitox, Denosumab, Docetaxel, Doxorubicin, Durvalumab, Epirubicin, Erlotinib, Estramustine, Etoposide, Everolimus, Floxuridine, Fludarabine, Fluorouracil, Fotemustine, Gefitinib, Gemcitabine, Gemtuzumab ozogamicin, Hydroxycarbamide, Ibritumomab tiuxetan, Idarubicin, Ifosfamide, Imatinib, Ipilimumab, Irinotecan, Isotretinoin, Ixabepilone, Lapatinib, Lenalidomide, Lomustine, Melphalan, Mercaptopurine, Methotrexate, Mitomycin, Mitoxantrone, Nedaplatin, Nelarabine, Nilotinib, Nivolumab, Ofatumumab, Oxaliplatin, Paclitaxel, Panitumumab, Panobinostat, Pazopanib, Pembrolizumab, Pemetrexed, Pentostatin, Pertuzumab, Pomalidomide, Ponatinib, Procarbazine, Raltitrexed, Regorafenib, Rituximab, Romidepsin, Ruxolitinib, Sorafenib, Streptozotocin, Sunitinib, Tamibarotene, Tegafur, Temozolomide, Temsirolimus, Teniposide, Thalidomide, Tioguanine, Topotecan, Tositumomab, Trastuzumab, Tretinoin, Valproate, Valrubicin, Vandetanib, Vemurafenib, Vinblastine, Vincristine, Vindesine, Vinflunine, Vinorelbine and Vorinostat.

The adsorbent and the anticancer agent of the invention may be used to treat or prevent a cancer or multiple cancers in a subject. In certain embodiments, the cancer may be one or a variant of a cancer selected from Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Extracranial Germ Cell Tumor, Extragonadal Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary (e.g., Metastatic), Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (T BC), Gestational Trophoblastic Tumor, Unknown Primary, Unusual Cancer of Childhood, Urethral Cancer, Uterine Cancer, Uterine Sarcoma, Waldenstrom Macroglobulinemia, and Wilms Tumor.

In particular, the cancer may be selected from:
tumours of epithelial origin affecting organs such as breast (breast adenocarcinoma), skin (melanoma), lung (non-small cell lung cancer and small cell lung cancer), kidney (renal cell carcinoma), pancreas (pancreatic carcinoma), bladder,
digestive tumours such as gastro-oesohagial adenocarcinomas,
head and neck cancers (in particular squamous tumors),
squamous lung tumours,
malignancies affecting blood of immune cells such as multiple myeloma, lymphoma (Hodgkin's and non-Hodgkin's of all types), leukemia among which lymphocytic leukemia (such as acute lymphoblastic leukemia (ALL), or chronic lymphocytic leukemia, (CLL)), myelogenous leukemia (such as acute myelogenous leukemia (AML), and chronic myelogenous leukemia (CML)), hairy cell leukemia, T-cell prolymphocytic leukemia, large granular lymphocytic leukemia, adult T-cell leukemia, adult T-cell lymphoma/leukemia.

In a particular embodiment, the cancer is selected from a cancer of the lung, a melanoma, a cancer of the pancreas, a cancer of the kidneys, refractory leukemia and lymphoma.

In certain embodiments, the method of the invention may further comprise administering one or more additional therapeutic agents conjointly with the anticancer agent. Representative therapeutic agents that may be conjointly administered with the anticancer agent include, without limitation: aminoglutethimide, amsacrine, anastrozole, asparaginase, AZD5363, Bacillus Calmette-Guerin vaccine (beg), bicalutamide, bleomycin, bortezomib, buserelin, busulfan, campothecin, capecitabine, carboplatin, carfilzomib, carmustine, chlorambucil, chloroquine, cisplatin, cladribine, clodronate, cobimetinib, colchicine, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, erlotinib, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, nilutamide, nocodazole, octreotide, olaparib, oxaliplatin, paclitaxel, pamidronate, pazopanib, pentostatin, perifosine, plicamycin, pomalidomide, porfimer, procarbazine, raltitrexed, rituximab, rucaparib, selumetinib, sorafenib, streptozocin, sunitinib, suramin, talazoparib, tamoxifen, temozolomide, temsirolimus, teniposide, testosterone, thalidomide, thioguanine, thiotepa, titanocene di chloride, topotecan, trametinib, trastuzumab, tretinoin, veliparib, vinblastine, vincristine, vindesine, and vinorelbine. Other representative therapeutic agents that may be conjointly administered with the anticancer agent include, without limitation, pemetrexed.

In a particular embodiment, anticancer therapy is a combination therapy with an immuno-oncology agent and at least one other anticancer agent. For example, the patient may be administered with an immuno-oncology agent and at least one other anticancer agent selected from platinum salts (such as cisplatin, carboplatin and the like), pemetrexed and etoposide. For example, the at least one other anticancer agent may be:
pemetrexed,
pemetrexed and platinum salts,
etoposide, or
etoposide and platinum salts.

In another embodiment, the present invention provides a kit, comprising an anticancer agent, and an adsorbent. In certain embodiments, the kit may be for use in treating a condition or disease as described herein.

The present invention provides a method of treating or preventing cancer, comprising conjointly administering an adsorbent and an anticancer agent. Thanks to the invention, administering the anticancer agent and the adsorbent provides improved efficacy relative to individual administration of the anticancer agent.

In certain embodiments, the anticancer agent is administered within about 5 minutes to within about 7 hours after the adsorbent. In a particular embodiment, the adsorbent is administered multiple times before the anticancer agent is administered in order to ensure that the anticancer immunosurveillance system of the patient is improved. For example, the adsorbent may be administered daily, one or several times a day, for several days. For example, the adsorbent may be administered daily, one or several times a day, at least 2, at least 3, at least 4, at least 5, at least 6 or at least 7 days before administration of the anticancer agent.

In certain aspects, the adsorbent is for use in a subject who has a cancer and who is administered, will be administered or has been administered with a substance, besides the anticancer agent, that may disturb the gut microbiota of said patient. Thanks to the invention, the deleterious impact of such substances may be prevented and thus the efficacy of the anticancer agent may be improved. Therefore, the invention relates to a method for mitigating the deleterious effects a substance may have on the gut microbiota of a subject suffering from cancer, said subject being the recipient of an anticancer agent therapy, comprising administering to said subject an effective amount of an adsorbent.

In certain embodiments, the substance is a pharmaceutical substance administered to treat a pathological condition in the patient. Indeed, certain pharmaceutical substances may be administered in order to treat a disease, but may have a deleterious effect on the gut microbiota when they reach the lower part of the intestine. The subject is still to receive the pharmaceutical substance for benefiting its desired effects but, on the other hand, solutions to avoid its secondary effects should be provided. Illustrative substances having this behavior include antibiotics. Antibiotics may be administered to a subject in order to treat a bacterial infection. However, since antibiotics are, by design, able to affect bacterial growth or survival, they threaten the gut microbiota balance and may induce dysbiosis when they reach the lower part of the intestine. This induced dysbiosis may in turn result in a decrease in the efficacy of an anticancer drug administered to the subject. Other illustrative pharmaceutical substances that may induce dysbiosis (also referred to as “dysbiosis-inducing pharmaceutical substances”) include, without limitation:
chemotherapy agents, such as taxanes (e.g. docetaxel, paclitaxel), anthracyclines (e.g. doxorubicin), topoisomerase inhibitors (e.g. etoposide, irinotecan), antimetabolites (e.g. methotrexate, cytarabine, 5-fluorouracil, gemcitabine, pemetrexed), alkylating agents (e.g. melphalan), kinase inhibitors (e.g. erlotinib),
antifungal agents, such as voroconazole, ambisome, posoconazole,
antiviral agents, such as acyclovir, methisazone,
anti-inflammatory agents, such as aspirin, ibuprofen; and
proton pump inhibitors such as omeprazole, pantoprazole, esomeprazole.

Accordingly, in another aspect of the invention the adsorbent is administered to a subject who has a cancer and who is treated, will be treated or has been administered with a dysbiosis-inducing pharmaceutical substance, such as an antibiotic.

Likewise, in certain aspects, the antibiotic-inactivating enzyme as described above is for use in a subject who has a cancer and who is administered, will be administered or has been administered with an antibiotic. In this aspect, the antibiotic-inactivating enzyme is selected among the enzymes able to inactivate the specific antibiotic administered to the subject (for example, a beta-lactamase is administered in case the antibiotic is a beta-lactam antibiotic; in another example, an erythromycin esterase is administered if the antibiotic is a macrolide antibiotic). Thanks to this aspect of the invention, the deleterious impact of the antibiotic may be prevented and thus the efficacy of the anticancer agent may be improved. Therefore, the invention relates to a method for mitigating the deleterious effects an antibiotic may have on the gut microbiota of a subject suffering from cancer, said subject being the recipient of an anticancer agent therapy, comprising administering to said subject an effective amount of an antibiotic-inactivating enzyme.

The adsorbent or the antibiotic-inactivating enzyme (if proper, because the dysbiosis-inducing pharmaceutical substance is an antibiotic) may be administered to the subject even long before initial administration of the anticancer agent. For example, the subject may have been diagnosed with a malignancy but the treatment could not begin before several days, weeks, months or years. In this case, should the subject suffer, between these events, from a disease that would need a treatment with a dysbiosis-inducing pharmaceutical agent, such as an antibiotic, it would be advantageous to prevent gut microbiota dysbiosis by administering an adsorbent or antibiotic-inactivating enzyme as provided herein. Likewise, the adsorbent or the antibiotic-inactivating enzyme may be administered to the subject even long before the start or after the end of administration of the anticancer agent. Firstly, it may unfortunately be that the subject's cancer could relapse. In this case, halting the systematic administration of an adsorbent or of an antibiotic-inactivating enzyme when the subject receives a dysbiosis-inducing pharmaceutical substance, such as an antibiotic, could severely impair the efficacy of a future therapy with the same or another anticancer agent. Secondly, some therapies, such as gene therapies, may be efficient several years after administration, as long as the therapeutic gene is expressed. In that case, the administration of the adsorbent or of the antibiotic-inactivating enzyme would be beneficial for improving this kind of long-lasting anticancer therapies. Of course, the adsorbent or the antibiotic-inactivating enzyme is preferably administered during the whole course of the anticancer agent therapy, when the subject is to receive a therapy with a dysbiosis-inducing pharmaceutical substance, such as an antibiotic.

In a particular embodiment, the invention relates to an adsorbent for improving the efficacy of an anticancer agent in a subject in need of such an anticancer agent, wherein the subject is also administered with a dysbiosis-inducing pharmaceutical substance, such as an antibiotic.

In another particular embodiment, the invention relates to an antibiotic-inactivating enzyme for improving the efficacy of an anticancer agent in a subject in need of such an anticancer agent, wherein the subject is also administered with an antibiotic

The invention also relates to an adsorbent for use in the prevention of the decrease of efficacy of an anticancer agent in a subject when said subject is administered with a dysbiosis-inducing pharmaceutical substance, such as an antibiotic.

The invention further relates to an antibiotic-inactivating enzyme for use in the prevention of the decrease of efficacy of an anticancer agent in a subject when said subject is administered with an antibiotic.

The invention also relates to an adsorbent for use to maintain the efficacy of an anticancer agent in a subject when said subject is administered with a dysbiosis-inducing pharmaceutical substance, such as an antibiotic.

Moreover, the invention also relates to an antibiotic-inactivating enzyme for use to maintain the efficacy of an anticancer agent in a subject when said subject is administered with an antibiotic.

The invention further relates to an adsorbent for use along with a dysbiosis-inducing pharmaceutical substance, such as an antibiotic, in a subject in need of an anticancer agent therapy.

In addition, the invention further relates to an antibiotic-inactivating enzyme for use along with an antibiotic in a subject in need of an anticancer agent therapy

The invention further relates to an adsorbent for use in combination with a dysbiosis-inducing pharmaceutical substance, such as an antibiotic, in a method for the treatment or prevention of a disease that may be treated or prevented with said dysbiosis-inducing pharmaceutical substance, wherein the subject in need of said treatment is also in need of an anticancer therapy.

The invention also relates to an antibiotic-inactivating enzyme for use in combination with an antibiotic for the treatment or prevention of a disease that may be treated or prevented with said antibiotic, wherein the subject in need of said treatment is also in need of an anticancer therapy.

The invention further relates to an adsorbent for use in a subject in need of an anticancer agent, for preventing the impact of a dysbiosis-inducing pharmaceutical substance, such as an antibiotic, on the efficacy of said anticancer agent.

In addition, the invention relates to an antibiotic-inactivating enzyme for use in a subject in need of an anticancer agent, for preventing the impact of an antibiotic on the efficacy of said anticancer agent.

The invention further relates to an adsorbent for use in a subject in need of an anticancer agent, for preventing the decrease in efficacy of said anticancer agent potentially induced by a dysbiosis-inducing pharmaceutical substance, such as an antibiotic, administered to said subject to treat or prevent another pathological condition that may be treated or prevented with said dysbiosis-inducing pharmaceutical substance.

The invention also relates to an antibiotic-inactivating enzyme for use in a subject in need of an anticancer agent, for preventing the decrease in efficacy of said anticancer agent potentially induced by an antibiotic administered to said subject to treat or prevent another pathological condition that may be treated or prevented with said antibiotic.

In a particular embodiment, the adsorbent or the antibiotic-inactivating enzyme is administered to the subject almost simultaneously with a dysbiosis-inducing pharmaceutical substance, for example an antibiotic. By “almost simultaneously”, it is meant that the adsorbent or the antibiotic-inactivating enzyme is administered shortly before, simultaneously, and/or shortly after administration of the dysbiosis-inducing pharmaceutical substance, in particular an antibiotic, preferably shortly before. In a particular embodiment, the adsorbent or the antibiotic-inactivating enzyme is administered less than 30 minutes before or after the dysbiosis-inducing pharmaceutical substance, in particular an antibiotic, has been administered, in particular less than 20 minutes, less than 19, 18, 17, 16, 15, 14, 13, 12, 11, 10, 9, 8, 7, 6, 5, 4, 3, 2 minutes, or less than one minute before or after the dysbiosis-inducing pharmaceutical substance, in particular an antibiotic, has been administered. In a further particular embodiment, the adsorbent or the antibiotic-inactivating enzyme is administered at least once a day, in particular at least twice a day, more particularly three times a day or four times a day. In a further particular embodiment, the adsorbent or the antibiotic-inactivating enzyme is administered during the whole course of the treatment with the dysbiosis-inducing pharmaceutical substance, in particular with an antibiotic. In a variant of this embodiment, the adsorbent or the antibiotic-inactivating enzyme may be administered a longer time than the dysbiosis-inducing pharmaceutical substance, in particular than an antibiotic, in order to ensure that any residual dysbiosis-inducing pharmaceutical substance, in particular any residual antibiotic, is eliminated. For example, the adsorbent or the antibiotic-inactivating enzyme may still be administered at least one day after, such as two days after interruption of the administration of the dysbiosis-inducing pharmaceutical substance, in particular after the administration of an antibiotic.

In a particular embodiment, the invention relates to an adsorbent or an antibiotic-inactivating enzyme for use in combination with an antibiotic, in particular almost simultaneously, to a subject who is in need of an anticancer agent. According to this embodiment, the adsorbent or the antibiotic-inactivating enzyme prevents the adverse effects the antibiotic could have on the intestinal microbiota of the subject, and therefore may improve the therapeutic efficacy of the anticancer agent.

Thus, the invention thus also relates to a kit comprising an adsorbent and a dysbiosis-inducing pharmaceutical substance, such as an antibiotic, or to a kit comprising or an antibiotic-inactivating enzyme and an antibiotic. The kit may be for use in the treatment or prevention of a pathological condition that may be treated or prevented with the dysbiosis-inducing pharmaceutical substance, such as an antibiotic. In a particular embodiment of the kit, the dysbiosis-inducing pharmaceutical substance is an antibiotic. The kit may further comprise instructions to implement the methods of the present invention, aiming at preventing the decrease in the efficacy of an anticancer agent. The components of the kit may be administered simultaneously, separately or sequentially. As provided above, the adsorbent or the antibiotic-inactivating enzyme may, in particular, be administered before, during, or after the administration of the dysbiosis-inducing pharmaceutical agent, such as an antibiotic, in particular shortly before or shortly after, more particularly shortly before.

### EXAMPLES

### Example 1

To evaluate the effect of antibiotic use during an anti-PD-1 treatment, mice are inoculated with cancer cells at Day 0. Mice are given an antibiotic from Day−14 to Day+25 by subcutaneous administration. After the inoculation of cancer cells, mice are treated with an anti-PD-1 treatment by intra peritoneal administration, twice a week, during two weeks. During the experiment, the tumour size is recorded every two days and the survival rate is measured as well. On Day+25, a larger tumour size is observed in mice receiving an antibiotic treatment compared to mice not receiving the antibiotic treatment.

### Example 2

To evaluate the effect of adsorbents administered with an antibiotic during an anti-PD-1 treatment, the same protocol as in example 1 is used, and an adsorbent is given by oral gavage, twice a day from Day−14 to Day+28. On Day+25, a smaller tumour size is observed in mice receiving the adsorbent 5 compared to mice receiving the antibiotic without the adsorbent.

### Example 3

To evaluate the effect of antibiotic-inactivating enzymes administered with antibiotics during an anti-PD-1 treatment, the same protocol as in example 1 is used, the antibiotic being a beta-lactam antibiotic. A beta-lactamase is also given by oral gavage, twice a day from Day−14 to Day+28. On Day+25, a smaller tumour size is observed in mice receiving the beta-lactamase compared to mice receiving the antibiotic without the beta-lactamase.

### Example 4

To evaluate the effect of antibiotic use during an anti-PD-L1 treatment, mice are inoculated with cancer cells at Day 0. Mice are given an antibiotic from Day−14 to Day+25 by subcutaneous administration. After the inoculation of cancer cells, mice are treated with an anti-PD-L1 treatment by intra peritoneal administration, twice a week, during two weeks. During the experiment, the tumour size is recorded every two days and the survival rate is measured as well. On Day+25, a larger tumour size is observed in mice receiving an antibiotic treatment compared to mice not receiving the antibiotic treatment.

### Example 5

To evaluate the effect of adsorbents administered with an antibiotic during an anti-PD-L1 treatment, the same protocol as in example 3 is used, and an adsorbent is given by oral gavage, twice a day from Day−14 to Day+28. On day+25, a smaller tumour size is observed in mice receiving the adsorbent compared to mice receiving the antibiotic without the adsorbent.

### Example 6

To evaluate the effect of antibiotic-inactivating enzymes administered with antibiotics during an anti-PD-L1 treatment, the same protocol as in example 4 is used, the antibiotic being a beta-lactam antibiotic. A beta-lactamase is also given by oral gavage, twice a day from Day-14 to Day+28. On day+25, a smaller tumour size is observed in mice receiving the beta-lactamase compared to mice receiving the antibiotic without the beta-lactamase.

### Example 7

To evaluate the effect of antibiotic use during an anti-CTLA-4 treatment, mice are inoculated with cancer cells at Day 0. Mice are given an antibiotic from Day−14 to Day+25 by subcutaneous administration. After the inoculation of cancer cells, mice are treated with an anti-CTLA-4 treatment by intra peritoneal administration, twice a week, during two weeks. During the experiment, the tumour size is recorded every two days and the survival rate is measured as well. On Day+25, a larger tumour size is observed in mice receiving an antibiotic treatment compared to mice not receiving the antibiotic treatment.

### Example 8

To evaluate the effect of adsorbents administered with an antibiotic during an anti-CTLA-4 treatment, the same protocol as in example 7 is used, and an adsorbent is given by oral gavage, twice a day from Day−14 to Day+28. On Day+25, a smaller tumour size is observed in mice receiving the adsorbent compared to mice receiving the antibiotic without the adsorbent.

### Example 9

To evaluate the effect of antibiotic-inactivating enzymes administered with antibiotics during an anti-CTLA-4 treatment, the same protocol as in example 1 is used, the antibiotic being a beta-lactam antibiotic. A beta-lactamase antibiotic-inactivating enzyme is also given by oral gavage, twice a day from Day−14 to Day+28. On Day+25, a smaller tumour size is observed in mice receiving the beta-lactamase compared to mice receiving the antibiotic without the beta-lactamase.

### Example 10

To evaluate the effect of antibiotic-inactivating enzymes administered with antibiotics during an anti-PD-1 treatment, the same protocol as in example 1 is used, the antibiotic being a macrolide. An erythromycin-esterase antibiotic-inactivating enzyme is also given by oral gavage, twice a day from Day−14 to Day+28. On Day+25, a smaller tumour size is observed in mice receiving the erythromycin-esterase compared to mice receiving the antibiotic without the erythromycin-esterase.

### Example 11

To evaluate the effect of antibiotic-inactivating enzymes administered with antibiotics during an anti-PD-L1 treatment, the same protocol as in example 1 is used, the antibiotic being a macrolide. An erythromycin-esterase antibiotic-inactivating enzyme is also given by oral gavage, twice a day from Day−14 to Day+28. On Day+25, a smaller tumour size is observed in mice receiving the erythromycin-esterase compared to mice receiving the antibiotic without the erythromycin-esterase.

### Example 12

To evaluate the effect of antibiotic-inactivating enzymes administered with antibiotics during an anti-CTLA-4 treatment, the same protocol as in example 1 is used, the antibiotic being a macrolide. An erythromycin-esterase antibiotic-inactivating enzyme is also given by oral gavage, twice a day from Day−14 to Day+28. On Day+25, a smaller tumour size is observed in mice receiving the erythromycin-esterase compared to mice receiving the antibiotic without the erythromycin-esterase.

### Example 13

To evaluate the effect of adsorbent use during an anti-PD-1 treatment, mice are inoculated with cancer cells at Day 0. After the inoculation of cancer cells, mice are treated with an anti-PD-1 treatment by intra peritoneal administration, twice a week, during two weeks. The mice are separated in two groups, one receiving an adsorbent given by oral gavage, twice a day from Day−14 to Day+28, and the other group not receiving an adsorbent. During the experiment, the tumour size is recorded every two days and the survival rate is measured as well. On Day+25, a larger tumour size is observed in mice not receiving the adsorbent compared to mice receiving the adsorbent.

### Example 14

To evaluate the effect of adsorbent use during an anti-PD-L1 treatment, mice are inoculated with cancer cells at Day 0. After the inoculation of cancer cells, mice are treated with an anti-PD-L1 treatment by intra peritoneal administration, twice a week, during two weeks. The mice are separated in two groups, one receiving an adsorbent given by oral gavage, twice a day from Day−14 to Day+28, and the other group not receiving an adsorbent. During the experiment, the tumour size is recorded every two days and the survival rate is measured as well. On Day+25, a larger tumour size is observed in mice not receiving the adsorbent compared to mice receiving the adsorbent.

### Example 15

To evaluate the effect of adsorbent use during an anti-CTLA-4 treatment, mice are inoculated with cancer cells at Day 0. After the inoculation of cancer cells, mice are treated with an anti-CTLA-4 treatment by intra peritoneal administration, twice a week, during two weeks. The mice are separated in two groups, one receiving an adsorbent given by oral gavage, twice a day from Day−14 to Day+28, and the other group not receiving an adsorbent. During the experiment, the tumour size is recorded every two days and the survival rate is measured as well. On Day+25, a larger tumour size is observed in mice not receiving the adsorbent compared to mice receiving the adsorbent.

### Example 16

To evaluate the capacity of adsorbent use during an anti-PD1 treatment, 60 mice were inoculated with Hepa 1-6 cells (5×10⁶) in the right front flank region. The date of tumor cell inoculation is denoted day 0. After tumor cell inoculation, the animals were checked daily for morbidity and mortality. During routine monitoring, the animals were checked for any effect of tumor growth and treatments on behavior such as mobility, food and water consumption, body weight gain/loss (body weights were measured twice per week after randomization), eye/hair matting and any other abnormalities. Mortality and observed clinical signs were recorded for individual animals in detail. Tumor volumes were measured twice per week in two dimensions using a caliper.

Mice were randomized in 3 groups of equal size:
Group A: anti-PD1 (3 mg/kg)+antibiotic placebo+adsorbent placebo
Group B: anti-PD1 (3 mg/kg)+clindamycin (25 mg/kg)+adsorbent placebo
Group C: anti-PD1 (3 mg/kg)+clindamycin (25 mg/kg)+adsorbent (1.5 g/kg)
Group D: anti-PD1 placebo (3 mg/kg)+antibiotic placebo+adsorbent placebo

The anti-PD1 used was RMP1-14 clone produced by Bioxcell. The isotype control of anti-PD1 (anti-PD1 placebo) was Rat IgG2a. The adsorbent was an activated charcoal. Clindamycin was obtained from GUANGZHOU BAIYUNSHAN TIANXIN PHARMACEUTICAL CO. The anti-PD1 was given twice a week for 3 weeks intraperitoneally. The clindamycin was given by subcutaneous route, every day, once a day, from D-14 to 4 days after the last injection of anti-PD-1. The adsorbent was administered by oral gavage, twice a day, from D-14 to 3 days after the last antibiotic injection.

We measured the size of the tumours in mice at day 29 and the median size for each group is reported in the table below:

| Group | Median tumour size |
|---|---|
| Group A: anti-PD1 + antibiotic placebo + | 536 mm³ |
| adsorbent placebo | |
| Group B: anti-PD1 + clindamycin + | 1613 mm³ |
| adsorbent placebo | |
| Group C: anti-PD1 + clindamycin + | 1142 mm³ |
| adsorbent | |
| Group D: anti-PD1 placebo + antibiotic | 1445 mm³ |
| placebo + adsorbent placebo | |

From the above table, it is clear that anti-PD1 was capable of reducing the growth of the tumor in group A compared with control group D. As mentioned in the present application, the addition of an antibiotic in group B provoked a loss of efficacy of the anti-PD1, the tumor growing as in control group D in the presence of the antibiotic. Surprisingly, administration of an adsorbent able to inactivate antibiotic residues (see group C) resulted in a restoration of the anti-PD1 efficacy. Therefore, we have shown that an adsorbent is able to improve the efficacy of anticancer agents in subjects administered with antibiotics.

1. A method for improving the efficacy of an anticancer agent in a subject in need of an anticancer treatment comprising administering an adsorbent to the subject, wherein the subject to be treated receives, will receive or has received a dysbiosis-inducing pharmaceutical agent.

2. The method of claim 1, wherein the dysbiosis-inducing pharmaceutical agent is an antibiotic administered for the prevention or the treatment of an infection.

3. The method of claim 1, wherein the adsorbent is activated charcoal.

4. The method of claim 1, wherein the adsorbent is in a formulation comprising:
a core containing an adsorbent, and
a layer of external coating formed around the core such that the adsorbent is released from the formulation in the lower part of the intestine.

5. The method of claim 4, wherein the core further comprises carrageenan, such as kappa-carrageenan.

6. A method for improving the efficacy of an anticancer agent in a subject in need of an anticancer therapy comprising administering an antibiotic-inactivating enzyme, wherein the subject receives, will receive or has received an antibiotic for the prevention or the treatment of an infection.

7. The method of claim 6, wherein (i) the enzyme is a beta-lactamase, in particular VIM-2 or ribaxamase, and the antibiotic is a beta-lactam antibiotic or (ii) the enzyme is an erythromycin esterase and the antibiotic is a macrolide.

8. The method of claim 6, wherein said enzyme is a hybrid protein molecule comprising two antibiotic-inactivating enzymes bonded together, said two enzymes inactivating the same or different antibiotics or antibiotics from the same or different classes.

9. The method of claim 6, formulated in a composition for oral administration suitable for the release of the antibiotic-inactivating enzyme in a desired part of the intestine, such as in the lower part of the intestine.

10. The method of claim 1, wherein the anticancer agent is selected from Afatinib, Aflibercept, Alemtuzumab, Alitretinoin, Altretamine, Anagrelide, Arsenic trioxide, Asparaginase, Atezolizumab, Avelumab, Axitinib, Azacitidine, Bendamustine, Bevacizumab, Bexarotene, Bleomycin, Bortezomib, Bosutinib, Busulfan, Cabazitaxel, Capecitabine, Carboplatin, Carmofur, Carmustine, Cetuximab, Chlorambucil, Chlormethine, Cisplatin, Cladribine, Clofarabine, Crizotinib, Cyclophosphamide, Cytarabine, Dacarbazine, Dactinomycin, Dasatinib, Daunorubicin, Decitabine, Denileukin diftitox, Denosumab, Docetaxel, Doxorubicin, Durvalumab, Epirubicin, Erlotinib, Estramustine, Etoposide, Everolimus, Floxuridine, Fludarabine, Fluorouracil, Fotemustine, Gefitinib, Gemcitabine, Gemtuzumab ozogamicin, Hydroxycarbamide, Ibritumomab tiuxetan, Idarubicin, Ifosfamide, Imatinib, Ipilimumab, Irinotecan, Isotretinoin, Ixabepilone, Lapatinib, Lenalidomide, Lomustine, Melphalan, Mercaptopurine, Methotrexate, Mitomycin, Mitoxantrone, Nedaplatin, Nelarabine, Nilotinib, Nivolumab, Ofatumumab, Oxaliplatin, Paclitaxel, Panitumumab, Panobinostat, Pazopanib, Pembrolizumab, Pemetrexed, Pentostatin, Pertuzumab, Pomalidomide, Ponatinib, Procarbazine, Raltitrexed, Regorafenib, Rituximab, Romidepsin, Ruxolitinib, Sorafenib, Streptozotocin, Sunitinib, Tam ibarotene, Tegafur, Temozolomide, Temsirolimus, Teniposide, Thalidomide, Tioguanine, Topotecan, Tositumomab, Trastuzumab, Tretinoin, Valproate, Valrubicin, Vandetanib, Vemurafenib, Vinblastine, Vincristine, Vindesine, Vinflunine, Vinorelbine and Vorinostat.

11. The method of claim 1, wherein the anticancer agent is an immuno-oncology agent.

12. The method of claim 11, wherein the immuno-oncology agent is selected from:
an immune checkpoint inhibitor, such as
a PD-1 inhibitor, e.g. nivolumab or pembrolizumab; or
a PD-L1 inhibitor, e.g. atezolizumab, avelumab, or durvalumab; or
a CTLA-4 inhibitor, e.g. ipilimumab; or
a PD-L2 inhibitor
a monoclonal antibody, e.g. trastuzumab;
a cancer vaccine, e.g. sipuleucel-T;
a non-specific immunotherapy, e.g. lenalidomide, interferons, or interleukins; and
chimeric antigen receptor (CAR)-T cell therapy, e.g. tisagenlecleucel, or axicabtagene ciloleucel.

13. The method of claim 1, wherein the anticancer agent is combined with at least one other anticancer agent, in particular with at least one immuno-oncology agent.

14. The method of claim 1, wherein the cancer is selected from Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Extracranial Germ Cell Tumor, Extragonadal Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary (e.g., Metastatic), Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (T BC), Gestational Trophoblastic Tumor, Unknown Primary, Unusual Cancer of Childhood, Urethral Cancer, Uterine Cancer, Uterine Sarcoma, Waldenstrom Macroglobulinemia, and Wilms Tumor.


SEQUENCE LISTING (90 sequences)

SEQ ID NO: 1 - protein, 240 aa, artificial
  VIM-2
VDSSGEYPTVSEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 2 - protein, 235 aa, artificial
  VIM-2 variant
MDSSGEYPTVSEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 3 - protein, 235 aa, artificial
  VIM-2 variant
MDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 4 - protein, 235 aa, artificial
  VIM-2 variant
VDSSGEYPTVSEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 5 - protein, 235 aa, artificial
  VIM-2 variant
VDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 6 - protein, 240 aa, artificial
  VIM-2  Q34R Q22H
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 7 - protein, 240 aa, artificial
  VIM-2  Q34R Q22N
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 8 - protein, 240 aa, artificial
  VIM-2  Q34R E130D
VDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 9 - protein, 240 aa, artificial
  VIM-2  Q34R E130D Q22N
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 10 - protein, 240 aa, artificial
  VIM-2  Q34R E130D Q22H
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 11 - protein, 235 aa, artificial
  VIM-2  Q34R Q22H DCT236
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 12 - protein, 235 aa, artificial
  VIM-2  Q34R Q22N DCT236
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 13 - protein, 235 aa, artificial
  VIM-2  Q34R E130D DCT236
VDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 14 - protein, 235 aa, artificial
  VIM-2  Q34R E130D Q22N DCT236
VDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 15 - protein, 235 aa, artificial
  VIM-2  Q34R E130D Q22H DCT236
VDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 16 - protein, 240 aa, artificial
  VIM-2  V1M Q34R Q22H
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 17 - protein, 240 aa, artificial
  VIM-2  V1M Q34R Q22N
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 18 - protein, 240 aa, artificial
  VIM-2  V1M Q34R E130D
MDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 19 - protein, 240 aa, artificial
  VIM-2  V1M Q34R E130D Q22N
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 20 - protein, 240 aa, artificial
  VIM-2  V1M Q34R E130D Q22H
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 21 - protein, 235 aa, artificial
  VIM-2  V1M Q34R Q22H DCT236
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 22 - protein, 235 aa, artificial
  VIM-2  V1M Q34R Q22N DCT236
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 23 - protein, 235 aa, artificial
  VIM-2  V1M Q34R E130D DCT236
MDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 24 - protein, 235 aa, artificial
  VIM-2  V1M Q34R E130D Q22N DCT236
MDSSGEYPTVSEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 25 - protein, 235 aa, artificial
  VIM-2  V1M Q34R E130D Q22H DCT236
MDSSGEYPTVSEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 26 - protein, 240 aa, artificial
  VIM-2 V10A
VDSSGEYPTASEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 27 - protein, 240 aa, artificial
  VIM-2 V10A Q34R Q22H
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 28 - protein, 240 aa, artificial
  VIM-2 V10A Q34R Q22N
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 29 - protein, 240 aa, artificial
  VIM-2 V10A Q34R E130D
VDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 30 - protein, 240 aa, artificial
  VIM-2 V10A Q34R E130D Q22N
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 31 - protein, 240 aa, artificial
  VIM-2 V10A Q34R E130D Q22H
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 32 - protein, 235 aa, artificial
  VIM-2 V10A Q34R Q22H DCT236
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 33 - protein, 235 aa, artificial
  VIM-2 V10A Q34R Q22N DCT236
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 34 - protein, 235 aa, artificial
  VIM-2 V10A Q34R E130D DCT236
VDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 35 - protein, 235 aa, artificial
  VIM-2 V10A Q34R E130D Q22N DCT236
VDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 36 - protein, 235 aa, artificial
  VIM-2 V10A Q34R E130D Q22H DCT236
VDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 37 - protein, 240 aa, artificial
  VIM-2 V10A V1M Q34R Q22H
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 38 - protein, 240 aa, artificial
  VIM-2 V10A V1M Q34R Q22N
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 39 - protein, 240 aa, artificial
  VIM-2 V10A V1M Q34R E130D
MDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 40 - protein, 240 aa, artificial
  VIM-2 V10A V1M Q34R E130D Q22N
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 41 - protein, 240 aa, artificial
  VIM-2 V10A V1M Q34R E130D Q22H
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 42 - protein, 235 aa, artificial
  VIM-2 V10A V1M Q34R Q22H DCT236
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 43 - protein, 235 aa, artificial
  VIM-2 V10A V1M Q34R Q22N DCT236
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 44 - protein, 235 aa, artificial
  VIM-2 V10A V1M Q34R E130D DCT236
MDSSGEYPTASEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 45 - protein, 235 aa, artificial
  VIM-2 V10A V1M Q34R E130D Q22N DCT236
MDSSGEYPTASEIPVGEVRLYNIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 46 - protein, 235 aa, artificial
  VIM-2 V10A V1M Q34R E130D Q22H DCT236
MDSSGEYPTASEIPVGEVRLYHIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLDGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTN

SEQ ID NO: 47 - protein, 263 aa, artificial
  P1A
EMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDDKLIAEATKVVMKALNMNGK

SEQ ID NO: 48 - protein, 215 aa, artificial
  P2A
ETGTISISQLNKNVWVHTELGYFNGEAVPSNGLVLNTSKGLVLVDSSWDNKLTKELIEMVEKKFQKRVTDVIITHAHADRIGGITALKERGIKAHSTALTAELAKNSGYEEPLGDLQTITSLKFGNTKVETFYPGKGHTEDNIVVWLPQYQILAGGCLVKSAEAKDLGNVADAYVNEWSTSIENVLKRYGNINSWPGHGEVGDKGLLLHTLDLLK

SEQ ID NO: 49 - protein, 258 aa, artificial
  P3A variants
  MISC_FEATURE (243)..(243): Xaa can be any naturally occuring amino acid
MKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDXKLIAEATKVVMKALN

SEQ ID NO: 50 - protein, 299 aa, artificial
  P3A variants
  misc_feature (280)..(280): Xaa can be any naturally occurring amino acid
  MISC_FEATURE (290)..(290): Xaa can be any naturally occuring amino acid
MIQKRKRTVSFRLVLMCTLLFVSLPITKTSAQASKTEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDXKLIAEATKVVMKALNMNGK

SEQ ID NO: 51 - protein, 293 aa, artificial
  KPC-1/2
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPSDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 52 - protein, 293 aa, artificial
  KPC-3
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKYSEAVIAAAARLALEGLGVNGQ

SEQ ID NO: 53 - protein, 292 aa, artificial
  KPC-4
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKHSEAVIAAAARLALEGLGVNGQ

SEQ ID NO: 54 - protein, 293 aa, artificial
  KPC-5
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 55 - protein, 292 aa, artificial
  KPC-6
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKHSEAVIAAAARLALEGLGVNGQ

SEQ ID NO: 56 - protein, 293 aa, artificial
  KPC-7
MSLYRRLVLLSCLSWPLAGFSATALTNLVAEPFAKLEQDFGGSIGVYAIDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRLRAAVPADWAVGDKTGTCGVYGTANDYAVVWPTGRAPIVLAVYTRAPNKDDKYSEAVIAAAARLALEGLGVNGQ

SEQ ID NO: 57 - protein, 292 aa, artificial
  KPC-8
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKYSEAVIAAAARLALEGLGVNGQ

SEQ ID NO: 58 - protein, 293 aa, artificial
  KPC-9
MSKYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGAYGTANDYAVVWPTGRAPIVLAVYTRAPNKDDKYSEAVIAAAARLALEGLGVNGQ

SEQ ID NO: 59 - protein, 293 aa, artificial
  KPC-10
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKYSEAVIAAAARLALEGLGVNGQ

SEQ ID NO: 60 - protein, 293 aa, artificial
  KPC-11
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVLWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 61 - protein, 293 aa, artificial
  KPC-12
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELEMNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 62 - protein, 293 aa, artificial
  KPC-13
MSLYRRLVLLSCLSWPLAGFSATALTNLVAEPFAKLEQDFGGSIGVYAMDTGSGATVSYRAEERFPLCSSFKGFLAAAVLARSQQQAGLLGTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKYSEAVIAAAARLALEGLGVNGQ

SEQ ID NO: 63 - protein, 291 aa, artificial
  KPC-14
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYANDYAVVWPTGRAPIVLAVYTRAPNKDDKHSEAVIAAAARLALEGLGVNGQ

SEQ ID NO: 64 - protein, 292 aa, artificial
  KPC-15
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVRWSPISEKYLTTGMTVLELSAAAVQYSDNAAANLLLKELGGPAKLTAFMRSIGDTTFRLDRWELELNSAIPGDARDTSSPRAVTESLQKLTLGSALAAPQRQQFVDWLKGNTTGNHRIRAAVPADWAVGDKTGTCGGYGTANDYAWWPTGRAPIVLAVYTRAPNKDDKYSEAVIAAAARLALEGLGVNGQ

SEQ ID NO: 65 - protein, 293 aa, artificial
  KPC-17
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQLVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 66 - protein, 269 aa, artificial
  NDM-1
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 67 - protein, 269 aa, artificial
  NDM-2
MELPNIMHPVAKLSTALAAALMLSGCMAGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 68 - protein, 269 aa, artificial
  NDM-3
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTNDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 69 - protein, 268 aa, artificial
  NDM-4
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGLVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 70 - protein, 270 aa, artificial
  NDM-5
MELPNIMHPVAKLSTALAAALMLSGCMPGEIRPTIGQQMETGDQRFGDLVFRQLAPNVWQHTSYLDMPGFGAVASNGLIVRDGGRVLLVDTAWTDDQTAQILNWIKQEINLPVALAVVTHAHQDKMGGMDALHAAGIATYANALSNQLAPQEGLVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 71 - protein, 269 aa, artificial
  NDM-6
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGMVAAQHSLTFAANGVWEPATAPNFGPLKVFYPGPGHTSDNITVGIDGTDIAFGGCLIKDSKAKSLGNLGDADTEHYAASVRAFGAAFPKASMIVMSHSAPDSRAAITHTARMADKLR

SEQ ID NO: 72 - protein, 268 aa, artificial
  NDM-7
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMNALHAAGIATYANALSNQLAPQEGLVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 73 - protein, 269 aa, artificial
  NDM-8
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMGALHAAGIATYANALSNQLAPQEGLVAAQHSLTFAANGVWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 74 - protein, 268 aa, artificial
  NDM-9
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQKGMVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 75 - protein, 269 aa, artificial
  NDM-10
MELPNIMHPVAKLSTALAAALMLSGCMPGEISPTIDQQMETGDQRFGDLVFRQLAPNVWQHTSYLDMPSFGAVTSNGLIVRDGGRVLWDTAWTDDQTAQILNWIKQEINLPVALAVVTHAHQDKMGGMDALHAAGIATYANALSNQLAPQEGMVAAQHSLTFAANGWVEPATAPNFGPLKVFYPGPGHTSDNITVGIDRTDIAFGGCLIKDSKAKSLGNLGDADTEHYAASARAFGAAFPKASMIVMSHSAPDSRAAITHTARMADKLR

SEQ ID NO: 76 - protein, 268 aa, artificial
  NDM-11
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAWTHAHQDKMGGMDALHAAGIATYANALSNQLAPQEGLVAAQHSLTFAANGWEPATAPNFGPLKVFYPGPGHTSDNITVGIDGTDIAFGGCLIKDSKAKSLGNLDDADTEHYAASARAFGAAFPKASMIVMSHSAPDSRAAITHTARMADKLR

SEQ ID NO: 77 - protein, 268 aa, artificial; identical to SEQ ID NO: 76
  NDM-12

SEQ ID NO: 78 - protein, 268 aa, artificial
  NDM-13
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTNDQTAQILNWIKQEINLPVALAWTHAHQDKMGGMDALHAAGIATYANALSNQLAPQEGLVAAQHSLTFAANGWEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 79 - protein, 260 aa, artificial
  P4A
EMKDDFAKLEEQFDAKLGIFALDTGTNRTVAYRPDERFAFASTIKALTVGVLLQQKSIEDLNQRITTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVGDKTGSGDYGTRNDIAIIWPPKGDPWLAVLSSRDKKDAKYDNKLIAEATKWMKALNMNGK

SEQ ID NO: 80 - protein, 299 aa, artificial
  P4A + peptide signal
MIQKRKRTVSFRLVLMCTLLFVSLPITKTSAQASKTEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVGDKTGSGDYGTRNDIAIIWPPKGDPVVLAVLSSRDKKDAKYDNKLIAEATKVVMKALNMNGK

SEQ ID NO: 81 - protein, 263 aa, Escherichia coli
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 82 - protein, 263 aa, Escherichia coli
QTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL

SEQ ID NO: 83 - protein, 257 aa, Staphylococcus aureus
KELNDLEKKYNAHIGVYALDTKSGKEVKFNSDKRFAYASTSKAINSAILLEQVPYNKLNKKVHINKDDIVAYSPILEKYVGKDITLKALIEASMTYSDNTANNKIIKEIGGIKKVKQRLKELGDKVTNPVRYEIELNYYSPKSKKDTSTPAAFGKTLNKLIANGKLSKENKKFLLDLMLNNKSGDTLIKDGVPKDYKVADKSGQAITYASRNDVAFVYPKGQSEPIVLVIFTNKDNKSDKPNDKLISETAKSVMKEF

SEQ ID NO: 84 - protein, 199 aa, Escherichia coli
MSNAKTKLGITKYSIVTNSNDSVTLRLMTEHDLAMLYEWLNRSHIVEWWGGEEARPTLADVQEQYLPSVLAQESVTPYIAMLNGEPIGYAQSYVALGSGDGRWEEETDPGVRGIDQLLANASQLGKGLGTKLVRALVELLFNDPEVTKIQTDPSPSNLRAIRCYEKAGFERQGTVTTPYGPAVYMVQTRQAFERTRSDA

SEQ ID NO: 85 - protein, 419 aa, Escherichia coli
MRFEEWVKDKHIPFKLNHPDDNYDDFKPLRKIIGDTRVVALGENSHFIKEFFLLRHTLLRFFIEDLGFTTFAFEFGFAEGQIINNWIHGQGTDDEIGRFLKHFYYPEELKTTFLWLREYNKAAKEKITFLGIDIPRNGGSYLPNMEIVHDFFRTADKEALHIIDDAFNIAKKIDYFSTSQAALNLHELTDSEKCRLTSQLARVKVRLEAMAPIHIEKYGIDKYETILHYANGMIYLDYNIQAMSGFISGGGMQGDMGAKDKYMADSVLWHLKNPQSEQKVIVVAHNAHIQKTPILYDGFLSCLPMGQRLKNAIGDDYMSLGITSYSGHTAALYPEVDTKYGFRVDNFQLQEPNEGSVEKAISGCGVTNSFVFFRNIPEDLQSIPNMIRFDSIYMKAELEKAFDGIFQIEKSSVSEVVYE

SEQ ID NO: 86 - protein, 388 aa, Bacteroides fragilis
MTMRIDTDKQMNLLSDKNVAIIGGGPVGLTMAKLLQQNGIDVSVYERDNDREARIFGGTLDLHKGSGQEAMKKAGLLQTYYDLALPMGVNIADKKGNILSTKNVKPENRFDNPEINRNDLRAILLNSLENDTVIWDRKLVMLEPGKKKWTLTFENKPSETADLVILANGGMSKVRKFVTDTEVEETGTFNIQADIHQPEINCPGFFQLCNGNRLMASHQGNLLFANPNNNGALHFGISFKTPDEWKNQTQVDFQNRNSVVDFLLKEFSDWDERYKELIHTTLSFVGLATRIFPLEKPWKSKRPLPITMIGDAAHLMPPFAGQGVNSGLVDALILSDNLADGKFNSIEEAVKNYEQQMFMYGKEAQEESTQNEIEMFKPDFTFQQLLNV

SEQ ID NO: 87 - protein, 267 aa, Enterococcus faecium
MLKQKELIANVKNLTESDERITACMMYGSFTKGEGDQYSDIEFYIFLKHSITSNFDSSNWLFDVAPYLMLYKNEYGTEVVIFDNLIRGEFHFLSEKDMNIIPSFKDSGYIPDTKAMLIYDETGQLENYLSEISGARPNRLTEENANFLLCNFSNLWLMGINVLKRGEYARSLELLSQLQKNTLQLIRMAEKNADNWLNMSKNLEKEISLENYKKFAKTTARLDKVELFEAYKNSLLLVMDLQSHLIEQYNLKVTHDILERLLNYISE

SEQ ID NO: 88 - protein, 532 aa, artificial
  TEM36-GGGGGG-CTXM16
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHWGGGGGGQTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL

SEQ ID NO: 89 - protein, 532 aa, artificial
  fusion CTXM16-6G-TEM36
QTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGLGGGGGGHPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 90 - protein, 537 aa, artificial
  fusion TEM36 - G(EAAAK)2 - CTXM16
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHWGEAAAKEAAAKQTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL